SEQUENCE LISTING (46 sequences)

SEQ ID NO: 1 - DNA, 51 bp, Homo sapiens
  variation (26)..(26): SNP rs10516486 y is c or t
gagttcagatcagctctatgaattaytaaatatctctcaaagcagatggga

SEQ ID NO: 2 - DNA, 51 bp, Homo sapiens
  variation (26)..(26): SNP rs10516483 s is c or g
ataagtttgaatgtggattgaataasagtgaattactaccaatcaatagga

SEQ ID NO: 3 - DNA, 51 bp, Homo sapiens
  variation (26)..(26): SNP rs1872701 k is g or t
tgtctctgttgtctttacttgtttgktctgcctgtaacatttgatacttcc

SEQ ID NO: 4 - DNA, 51 bp, Homo sapiens
  variation (26)..(26): SNP rs10496637 k is t or g
ttgctaaatattaagaaaatcttgaktcacacaaataagctgcccactgat

SEQ ID NO: 5 - DNA, 51 bp, Homo sapiens
  variation (26)..(26): SNP rs950357 m is a or c
attctggaaaatgtttgctttgggcmgacccagactggcattcgatatctg

SEQ ID NO: 6 - DNA, 51 bp, Homo sapiens
  variation (26)..(26): SNP rs10516928 k is t or g
tctcttaaccattctgctatactgcktttcacaaaaatgacacacactttt

SEQ ID NO: 7 - DNA, 51 bp, Homo sapiens
  variation (26)..(26): SNP rs1342337 r is a or g
aacagtggtacctaatgactccctargcctcaaattatattaaaagacaat

SEQ ID NO: 8 - DNA, 51 bp, Homo sapiens
  variation (26)..(26): SNP rs1937840 s is c or g
tttctatctcttccttaggaaactgsatagattaatgcacaagcaaggaaa

SEQ ID NO: 9 - DNA, 51 bp, Homo sapiens
  variation (26)..(26): SNP rs10505774 y is c or t
tctgttccagtctacatactttttayggaactacaaatataaataagctct

SEQ ID NO: 10 - DNA, 51 bp, Homo sapiens
  variation (26)..(26): SNP rs2302733 r is a or g
acctgtaaccttctcaatggcaccaraaacaacggcactgaccctggacac

SEQ ID NO: 11 - DNA, 51 bp, Homo sapiens
  variation (26)..(26): SNP rs738981 y is c or t
agattgatagctatcaggaaatcttygtatgtatgaatctctcacaagtct

SEQ ID NO: 12 - DNA, 51 bp, Homo sapiens
  variation (26)..(26): SNP rs6683832 r is g or a
atttaatacaagattcttaaacttgrttctgtctctattatttaatttcta

SEQ ID NO: 13 - DNA, 51 bp, Homo sapiens
  variation (26)..(26): SNP rs2300166 r is g or a
tgataacagcagctccattttctacrcagggaagttgggataatcaaataa

SEQ ID NO: 14 - DNA, 51 bp, Homo sapiens
  variation (26)..(26): SNP rs1901765 y is t or c
caggcctaaaactgcttattaaacaygagatcctgaccttctctaacacac

SEQ ID NO: 15 - DNA, 51 bp, Homo sapiens
  variation (26)..(26): SNP rs1401385 w is t or a
acaaaggaatgcttgccatagatagwcaatttgccttaagatacctcattt

SEQ ID NO: 16 - DNA, 51 bp, Homo sapiens
  variation (26)..(26): SNP rs1717045 r is a or g
cttagccttacttgtgccttattctrttctttaactatcacttatgctgca

SEQ ID NO: 17 - DNA, 51 bp, Homo sapiens
  variation (26)..(26): SNP rs790837 r is g or A
ataaattatgtggtgaaaaaagtacrggactggaaagcaacagatctgggt

SEQ ID NO: 18 - DNA, 51 bp, Homo sapiens
  variation (26)..(26): SNP rs10484396 y is t or c
ttcccctcttttctgcactcagcaaygttaacctatgtccctctctggatg

SEQ ID NO: 19 - DNA, 51 bp, Homo sapiens
  variation (26)..(26): SNP rs10485136 y is t or c
ctacactttttctcatcctctctctytgttaaaggcatcatcacattccta

SEQ ID NO: 20 - DNA, 51 bp, Homo sapiens
  variation (26)..(26): SNP rs9294364 r is g or a
tggatgtcccttctactttttccatrcataataaaaccaaacaaaactgta

SEQ ID NO: 21 - DNA, 51 bp, Homo sapiens
  variation (26)..(26): SNP rs881278 r is a or g
ctaattcatcttactcatattatgtrttaaaaacagtggcacttcagttta

SEQ ID NO: 22 - DNA, 51 bp, Homo sapiens
  variation (26)..(26): SNP rs720613 y is c or t
gtagaaaggttgacagtgtactgaaygatgcaggctatcttcacccaactt

SEQ ID NO: 23 - DNA, 51 bp, Homo sapiens
  variation (26)..(26): SNP rs1478895 s is c or g
ccatggtacatttgccagaactaagsagtaattgttaccacaatattagcg

SEQ ID NO: 24 - DNA, 51 bp, Homo sapiens
  variation (26)..(26): SNP rs2289965 y is c or t
gtcctcagcatctgtcaagaaactgygtgtctggtatttggtcctcagctg

SEQ ID NO: 25 - DNA, 51 bp, Homo sapiens
  variation (26)..(26): SNP rs2289965 y is c or t
gtgcttgcatcccgcttcatgatgaygtagtgagcctcaccgtcctcctgc

SEQ ID NO: 26 - DNA, 51 bp, Homo sapiens
  variation (26)..(26): SNP rs10502263 y is t or c
atgattcaagggtacaatgtggtcaygaaaatggaagacagtgtcaccaag

SEQ ID NO: 27 - DNA, 51 bp, Homo sapiens
  variation (26)..(26): SNP rs1049380 k is t or g
gttattttaactcagaaaacatactkgcattaagctcttgagcctcagaat

SEQ ID NO: 28 - DNA, 51 bp, Homo sapiens
  variation (26)..(26): SNP rs10506140 r is g or a
tgaactggataagaaaaaaaattcartattcaaagagcatgatattccctt

SEQ ID NO: 29 - DNA, 51 bp, Homo sapiens
  variation (26)..(26): SNP rs10507393 y is t or c
ctatgctcttactaggagttatggtyctttttatgtcttagatgatgcttg

SEQ ID NO: 30 - DNA, 51 bp, Homo sapiens
  variation (26)..(26): SNP rs10508021 s is g or c
taactccctagccatatactcttaastaagctgaaggcaagcagggccttc

SEQ ID NO: 31 - DNA, 51 bp, Homo sapiens
  variation (26)..(26): SNP rs1886560 y is c or t
tgttttttgaatccagctcgtaaagyctataattaggaggaagcatcaaag

SEQ ID NO: 32 - DNA, 51 bp, Homo sapiens
  variation (26)..(26): SNP rs2165739 r is a or g
taactctgctactgattatctttgcratttttaggaagtgtaccattcttt

SEQ ID NO: 33 - DNA, 24 bp, Artificial sequence
  BANK1 isoform forward primer
tcaaagcagatgggagatctcaac

SEQ ID NO: 34 - DNA, 25 bp, Artificial sequence
  BANK1 isoform reverse primer
cacatggaatttcagtgggaagcac

SEQ ID NO: 35 - DNA, 22 bp, Artificial sequence
  BLK forward primer
acggcccaagagggggccaagt

SEQ ID NO: 36 - DNA, 23 bp, Artificial sequence
  BLK reverse primer
gttgctcatccctgggtatggca

SEQ ID NO: 37 - DNA, 26 bp, Artificial sequence
  IPTR2 forward primer
tggctcaaatgattgtggagaagaat

SEQ ID NO: 38 - DNA, 26 bp, Artificial sequence
  IPTR2 reverse primer
actgatgaaaggctagtcacggcttc

SEQ ID NO: 39 - DNA, 22 bp, Artificial Sequence
  primer f-BLK
caccatggggctggtaagtagc

SEQ ID NO: 40 - DNA, 20 bp, Artificial Sequence
  primer r-BLK
gggctgcagctcgtactgcc

SEQ ID NO: 41 - DNA, 23 bp, Artificial sequence
  primer f-BANK
caccatgctgccagcagcgccag

SEQ ID NO: 42 - DNA, 30 bp, Artificial Sequence
  primer r-BANK
ataataaccttctttaatgatctttcttgc

SEQ ID NO: 43 - DNA, 29 bp, Artificial Sequence
  primer f-FLAG-k
cacaaccatggattacaaggatgacgacg

SEQ ID NO: 44 - DNA, 30 bp, Artificial Sequence
  primer f-FLAG-m
attacaaggatgacgacgataagatgctgc

SEQ ID NO: 45 - DNA, 29 bp, Artificial sequence
  primer f-FLAG-BANK
cgacgataagatgctgccagcagcgccag

SEQ ID NO: 46 - DNA, 24 bp, Artificial sequence
  primer r-BANK-H1
aggatccttctttaatgatctttc

(19) United States
(12) Patent Application Publication
(10) Pub. No.: US 2011/0319288 A1
(43) Pub. Date: Dec. 29, 2011

(54) BANK1 RELATED SNPS AND SLE AND/OR MS SUSCEPTIBILITY

(75) Inventors: Jérôme Wojcik, Divonne Les Bains (FR); Marta Alarcôn-Riquelme, Stockholm (SE); Casimiro Castillejo-López, Uppsala (SE)

(73) Assignee: MERCK SERONO S.A., COINSINS, VAUD (CH)

(21) Appl. No.: 13/147,377

(22) PCT Filed: Mar. 1, 2010
(86) PCT No.: PCT/EP10/52554

§ 371 Date: Aug. 2, 2011

Related U.S. Application Data
(60) Provisional application No. 61/156,970, filed on Mar. 3, 2009.

(30) Foreign Application Priority Data
Mar. 3, 2009 (EP) 09154190.4

(51) Int. Cl. C40B 30/04; C12Q 1/68

(52) U.S. Cl. 506/9; 435/6.12; 435/6.11

(57) ABSTRACT

The invention relates to a method of genotyping and for predicting the susceptibility for SLE and/or MS by using SNPs related to BANK1 alone or in combination with at least one other SNP.

### FIELD OF THE INVENTION

The invention relates to BANK1, SNPs (single nucleotide polymorphisms) related to BANK1, combinations of BANK1 SNPs with other SNPs and their use in the prediction of SLE (Systemic Lupus Erythematosus) and/or MS (Multiple Sclerosis).

### BACKGROUND OF THE INVENTION

Genetic techniques allow the identification of single nucleotide polymorphisms (SNPs) in individuals. SNPs are changes in a gene in one single nucleotide and the identification of SNPs can be correlated with a biological pathway having implications for a particular disease. The polymorphisms may be correlated also with a predisposition or risk for a disease by application of statistical analyses. Accordingly, targeting a particular biological pathway related to a disease is a means to treat such disease.

B-cell scaffold protein with ankyrin repeats (BANK1) is expressed in B cells and is tyrosine phosphorylated upon B-cell antigen receptor (BCR) stimulation. The BANK1 gene has 284 kb. BANK1 is an adaptor protein (14, 15) expressed mainly in B cells. The two full length isoforms of 785 and 755 amino acids, differ by 30 amino acids in the N-terminal region coded by the alternative exon 1A and contain ankyrin repeat motifs and coiled-coil regions—structures highly similar between BANK1, BCAP and D of adaptor proteins (16). B cell activation through BCR engagement leads to tyrosine phosphorylation of BANK1, which in turn promotes its association with the protein tyrosine kinase Lyn and the calcium channel IP3R (4). BANK1 serves as a docking station bridging together and facilitating phosphorylation and activation of IP3R by Lyn and the consequent release of Ca²⁺ from endoplasmic reticulum stores (4, 17).

BANK1 and the pathway it is involved in, is considered to have implications for inflammatory and auto-immune disorders. In particularly, BANK1 is expressed in B-cells and therefore the pathway wherein BANK1 is involved has an implication for diseases associated with B-cells, e.g. Systemic Lupus Erythematosus (SLE). Multiple Sclerosis (MS) is related to T-cells, however, also the role of B-cells has been discussed in this disease. Accordingly, polymorphisms in the BANK1 gene may be used to diagnose a predisposition or risk for MS. Moreover, the BANK1 pathway may have implications for MS. In consequence, targeting this pathway and its modulation may represent a means to prevent or treat MS.

A number of genes associated with complex diseases like SLE or MS have been identified, but their individual contribution to genetic susceptibility is small. Genetic epistatic interactions might explain larger risk effects and reveal biological pathways.

### SUMMARY OF THE INVENTION

According to one aspect of the invention, a method is provided for diagnosing an individual for the predisposition of, the risk of developing or suffering from an auto-immune or inflammatory disease wherein the pathway of BANK1 is involved.

According to another aspect of the invention, a method is provided for diagnosing an individual for the predisposition of, the risk of developing or suffering from an auto-immune or inflammatory disease wherein a SNP in Linkage Disequilibrium (LD) with one BANK1 SNP can be used and preferably at least one BANK1 SNP is combined with at least one second SNP.

### DETAILED DESCRIPTION OF THE INVENTION

The following is a brief description of the Figures:

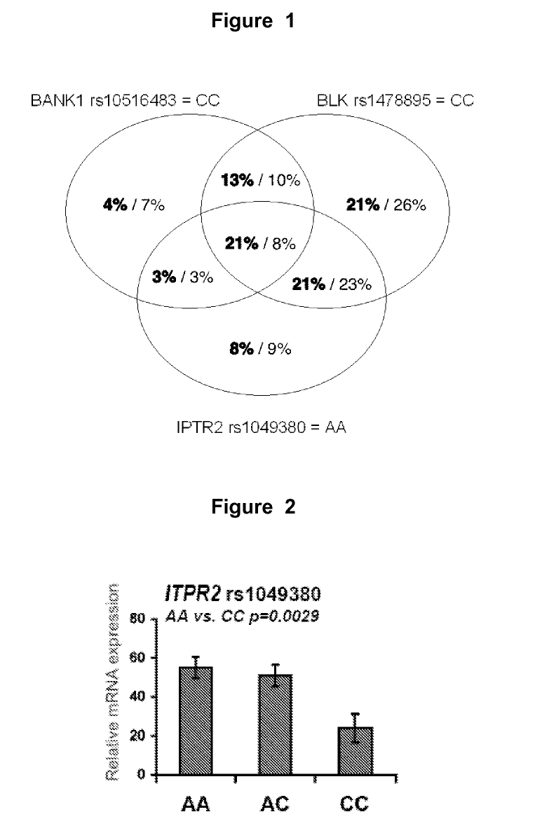

FIG. 1 Venn diagram displaying the proportions x/y of cases (x, in bold) and controls (y) having each risk allele for BANK1 (rs10516483), BLK (rs1478895) and ITPR2 (rs1049380).

FIG. 2 Correlations of the levels of ITPR2 with genotypes of the 3′ UTR SNP rs1049380. Relative mRNA levels reflect mRNA abundance of the transcripts normalized to the level of TBP.

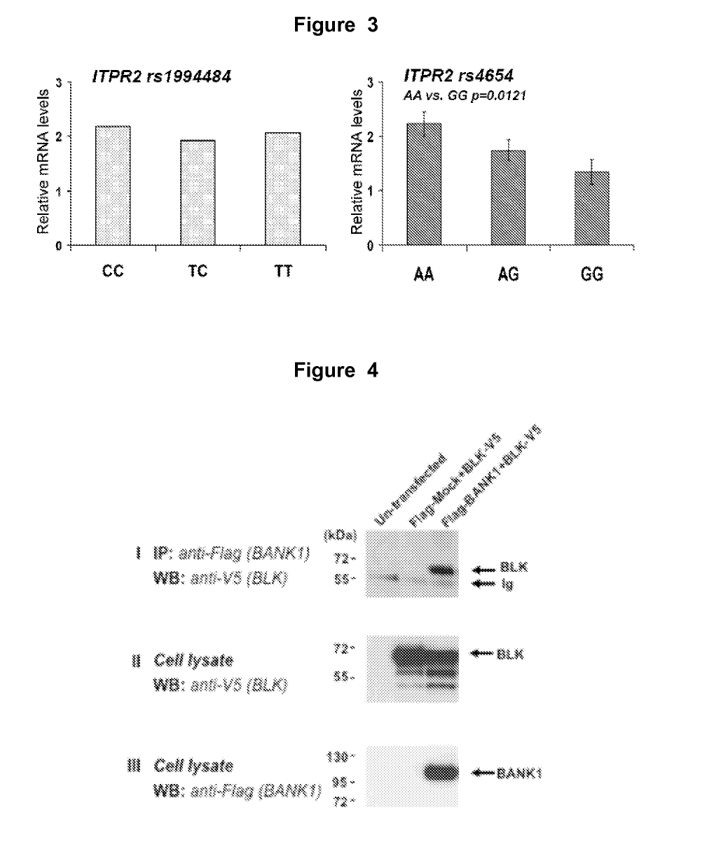

FIG. 3 Correlations of the levels of ITPR2 with genotypes of the 3′ UTR SNP rs4654 (in linkage disequilibrium with rs1049380, FIG. 1), while another SNP rs1994484 outside of the 3′ UTR region shows no correlation. Relative mRNA levels reflect mRNA abundance of the transcripts normalized to the level of TBP.

FIG. 4 Immunoprecipitation and western blot showing the physical interaction between BANK1 and BLK. BANk1-FLAG and BLK-V5 were co-transfected onto HEK293T cells and immunoprecipitation was done using anti-FLAG antibodies. Western blot was performed using anti-V5 antibodies and confirmed with anti-FLAG antibodies. Lanes show: 1. Untransfected cells; 2. FLAG mock and BLK transfection only; and 3. Co-transfection of FLAG-BANK1 and BLK-V5.

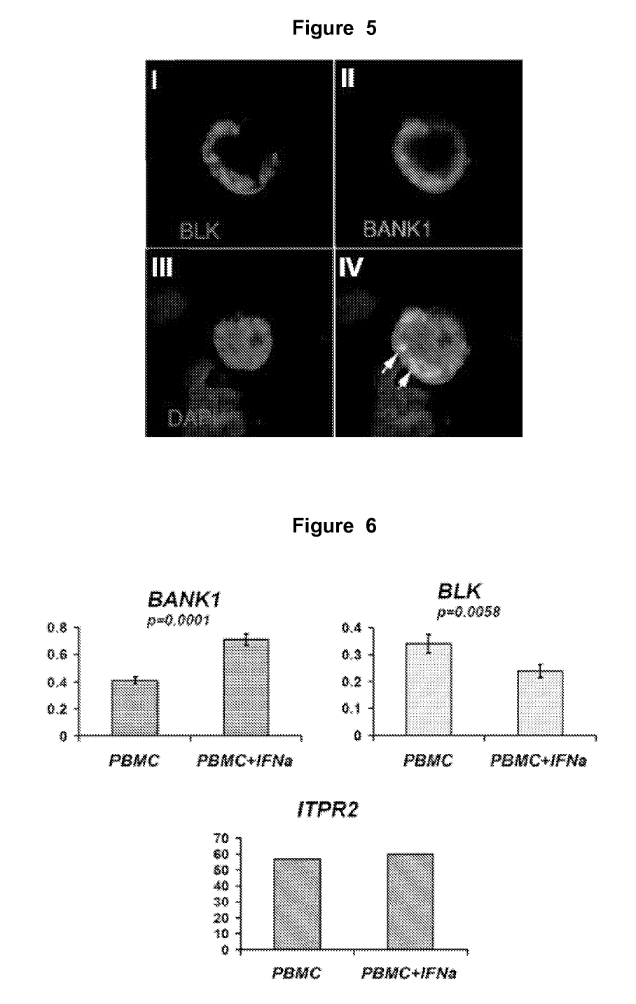

FIG. 5 Cellular co-localization of BANK1 and BLK. HEK293T kidney cells were co-transfected with constructs containing BLK-GFP (I) and BANK1 detected with anti-human BANK1 polyclonal antibodies (II). DAPI was used to recognize the nucleus of the cells (III). BLK localizes to the plasma membrane and the cytoplasm, while BANK1 is localized in the cytoplasm. Merging shows co-localization of BANK1 and BLK within sub-cellular vesicles in the cytoplasmic compartment (IV) as shown by the arrows.

FIG. 6 Effect of interferon-α stimulation of PBMCs on the transcript expression levels of BANK1, BLK and ITPR2. PBMCs were stimulated with 1000 U/ml of IFNα (Raybiotech) for 6 hours in culture followed by total RNA purification and qRT-PCR analysis.

The invention relates to a method for genotyping comprising the steps of:
a. using a nucleic acid isolated from a sample of an individual; and
b. determining the type of nucleotide in rs10516486, rs10516483, rs1872701, rs10496637, rs950357, rs10516928, rs1342337, rs1937840, rs10505774, rs2302733, rs738981, rs6683832, rs2300166, rs1901765, rs1401385, rs1717045, rs790837, rs10484396, rs10485136, rs9294364, rs881278, rs720613, rs1478895, rs1992529, rs2289965, rs10502263, rs1049380, rs10506140, rs10507393, rs10508021, rs1886560, rs2165739 and/or rs10508021 in the diallelic marker, and/or in a SNP in Linkage Disequilibrium (LD) with one or more of these SNPs, and/or one or more SNP in LD with either of BANK1, BLK and/or ITPR2.

In another aspect the invention relates to a method for genotyping comprising the steps of:
a. using a nucleic acid isolated from a sample of an individual;
b. determining the type of nucleotide in:
   rs10516486 and rs950357,
   rs10516486 and rs1342337,
   rs10516486 and rs1937840,
   rs10516483 and rs1401385,
   rs10516483 and rs1717045,
   rs10516483 and rs1478895,
   rs10516483 and rs1049380,
   rs10516483 and rs10507393,
   rs10516483 and rs10508021,
   rs1872701 and rs10508021, or
   rs10516483, rs1478895 and rs1049830 in the diallelic marker, or in a SNP in Linkage Disequilibrium (LD) with one or more of these SNPs or one or more SNP in LD with either of BANK1, BLK and/or ITPR2; and
c. correlating the results of step b. with a risk of susceptibility for Systemic Lupus Erythematosus (SLE).

In the method according to the invention the identity of the nucleotides at said diallelic markers is preferably determined for both copies of said diallelic markers present in said individual's genome.

The method for genotyping according to the invention is preferably performed by a microsequencing assay. The method preferably further comprises amplifying a portion of a sequence comprising the diallelic marker prior to said determining step. Preferably said amplifying is performed by PCR. The method according to the invention further comprises the step of correlating the result of the genotyping steps with a risk of suffering or a predisposition for an auto-immune disease or inflammatory disease.

In a preferred method of the invention the method further comprises the step of correlating the result of the genotyping steps with a risk of susceptibility for Systemic Lupus Erythematosus (SLE) and/or Multiple Sclerosis (MS).

Particularly useful SNPs and SNP combinations have been identified which are depicted in Table 1. Table 1 shows advantageous SNP combinations and their risk alleles for MS and/or SLE.

The sequences of preferred SNPs are depicted in the following and in the sequence listing contained at the end of the application text.

| | | SEQ | |
|---|---|---|---|
| SNP | Context Sequence | ID NO. | Allele |
| rs10516486 | gagttcagatcagctctatgaattaYtaaatatctctcaaagcagatggga | 1 | C or T |
| rs10516483 | ataagtttgaatgtggattgaataaSagtgaattactaccaatcaatagga | 2 | C or G |
| rs1872701 | tgtctctgttgtctttacttgtttgKtctgcctgtaacatttgatacttcc | 3 | G or T |
| rs10496637 | ttgctaaatattaagaaaatcttgaKtcacacaaataagctgcccactgat | 4 | T or G |
| rs950357 | attctggaaaatgtttgctttgggcMgacccagactggcattcgatatctg | 5 | A or C |
| rs10516928 | tctcttaaccattctgctatactgcKtttcacaaaaatgacacacactttt | 6 | T or G |
| rs1342337 | aacagtggtacctaatgactccctaRgcctcaaattatattaaaagacaat | 7 | A or G |
| rs1937840 | tttctatctcttccttaggaaactgSatagattaatgcacaagcaaggaaa | 8 | C or G |
| rs10505774 | tctgttccagtctacatactttttaYggaactacaaatataaataagctct | 9 | C or T |
| rs2302733 | acctgtaaccttctcaatggcaccaRaaacaacggcactgaccctggacac | 10 | A or G |
| rs738981 | agattgatagctatcaggaaatcttYgtatgtatgaatCTCTCACAAGTCT | 11 | C or T |
| rs6683832 | atttaatacaagattcttaaacttgRttctgtctctattatttaatttcta | 12 | G or A |
| rs2300166 | tgataacagcagctccattttctacRcagggaagttgggataatcaaataa | 13 | G or A |
| rs1901765 | caggcctaaaactgcttattaaacaYgagatcctgaccttctctaacacac | 14 | T or C |
| rs1401385 | acaaaggaatgcttgccatagatagWcaatttgccttaagatacctcattt | 15 | T or A |
| rs1717045 | cttagccttacttgtgccttattctRttctttaactatcacttatgctgca | 16 | A or G |
| rs790837 | ataaattatgtggtgaaaaaagtacRggactggaaagcaacagatctgggt | 17 | G or A |
| rs10484396 | ttcccctcttttctgcactcagcaaYgttaacctatgtccctctctggatg | 18 | T or C |
| rs10485136 | ctacactttttctcatcctctctctYtgttaaaggcatcatcacattccta | 19 | T or C |
| rs9294364 | tggatgtcccttctactttttccatRcataataaaaccaaacaaaactgta | 20 | G or A |
| rs881278 | ctaattcatcttactcatattatgtRttaaaaacagtggcacttcagttta | 21 | A or G |
| rs720613 | gtagaaaggttgacagtgtactgaaYgatgcaggctatcttcacccaactt | 22 | C or T |
| rs1478895 | ccatggtacatttgccagaactaagSagtaattgttaccacaatattagcg | 23 | C or G |
| rs1992529 | gtcctcagcatctgtcaagaaactgYgtgtctggtatttggtcctcagctg | 24 | C or T |
| rs2289965 | gtgcttgcatcccgcttcatgatgaYgtagtgagcctcaccgtcctcctgc | 25 | C or T |
| rs10502263 | atgattcaagggtacaatgtggtcaYgaaaatggaagacagtgtcaccaag | 26 | T or C |
| rs1049380 | gttattttaactcagaaaacatactKgcattaagctcttgagcctcagaat | 27 | T or G |
| rs10506140 | tgaactggataagaaaaaaaattcaRtattcaaagagcatgatattccctt | 28 | G or A |
| rs10507393 | ctatgctcttactaggagttatggtYctttttatgtcttagatgatgcttg | 29 | T or C |
| rs10508021 | taactccctagccatatactcttaaStaagctgaaggcaagcagggccttc | 30 | G or C |
| rs1886560 | tgttttttgaatccagctcgtaaagYctataattaggaggaagcatcaaag | 31 | C or T |
| rs2165739 | taactctgctactgattatctttgcRatttttaggaagtgtaccattcttt | 32 | A or G |

IUPAC SNP codes:

| IUPAC Code | SNP |
|---|---|
| R | G or A |
| Y | T or C |
| M | A or C |
| K | G or T |
| S | G or C |
| W | A or T |

In the above described method according to the invention the presence of a C or a T in rs10516486, a C or a G in rs10516483, a G or a T in rs1872701, a T or a G in rs10496637, a A or C in rs950357, a T or a G in rs10516928, a A or a G in rs1342337, a C or a G in rs1937840, a C or a T in rs10505774, a A or a G in rs2302733, a C or a T in rs738981, a G or a A in rs6683832, a G or a A in rs2300166, a T or a C in rs1901765, a T or a A in rs1401385, a A or a G in rs1717045, a G or a A in rs790837, a T or a C in rs10484396, a T or a C in rs10485136, a G or a A in rs9294364, a A or a G in rs881278, a C or a T in rs720613, a C or a G in rs1478895, a C or a T in rs1992529, a C or a T in rs2289965, a T or a C in rs10502263, a T or a G in rs1049380, a G or a A in rs10506140, a T or a C in rs10507393, a G or a C in rs10508021, a C or a T in rs1886560, and/or a A or a G in rs2165739 in said individual indicates that said individual has a risk of susceptibility to SLE and/or MS. In the enumeration above, the risk allele is listed first (i.e. if it is mentioned “presence of a X or a Y”, the risk allele is X).

In particular, in the above described method according to the invention the presence of a C or a T in rs10516486, a C or a G in rs10516483, a G or a T in rs1872701, a A or C in rs950357, a A or a G in rs1342337, a C or a G in rs1937840, a T or a A in rs1401385, a A or a G in rs1717045, a C or a G in rs1478895, a T or a G in rs1049380, a T or a C in rs10507393, and/or a G or a C in rs10508021 in said individual indicates that said individual has a risk of susceptibility to SLE. In the enumeration above, the risk allele is listed first.

In another aspect the invention relates to one or more SNPs selected from the group consisting of rs10516486, rs10516483, rs1872701, rs10496637, rs950357, rs10516928, rs1342337, rs1937840, rs10505774, rs2302733, rs738981, rs6683832, rs2300166, rs1901765, rs1401385, rs1717045, rs790837, rs10484396, rs10485136, rs9294364, rs881278, rs720613, rs1478895, rs1992529, rs2289965, rs10502263, rs1049380, rs10506140, rs10507393, rs10508021, rs1886560 and/or rs2165739, SNPs in Linkage Disequilibrium (LD) with one or more of these SNPs, and one or more SNPs in LD with either of BANK1, BLK and/or ITPR2 for use in predicting that an individual has a risk of susceptibility for SLE and/or for MS.

In another aspect the invention relates to at least two SNPs selected from the group consisting of rs10516486, rs950357, rs1342337, rs1937840, rs10516483, rs1401385, rs1717045, rs1478895, rs1049380, rs10507393, rs10508021, rs1872701, SNPs in Linkage Disequilibrium (LD) with one or more of these SNPs, and one or more SNPs in LD with either of BANK1, BLK and/or ITPR2 for use in predicting that an individual has a risk of susceptibility for SLE.

One example of a SNP that is in LD with a gene identified to be useful in the invention and/or one SNP identified by the inventors is rs4654 (ITPR2). It could be shown that rs4654 is in LD with SNP rs1049380 (see FIGS. 1 and 3). Hence it represents an example of SNPs in LD with genes and SNPs that can be identified according to the procedure of the current invention.

Particular useful is a combination of rs10516486 with rs10496637, rs950357, rs10516928, rs1342337, rs1937840, rs10505774, rs2302733 and/or rs738981; or rs10516483 with rs6683832, rs2300166, rs1901765, rs1401385, rs1717045, rs790837, rs10484396, rs10485136, rs9294364, rs881278, rs720613, rs1478895, rs1992529, rs2289965, rs10502263, rs1049380, rs10506140, rs10507393, rs10508021 and/or rs1886560; or rs1872701 with rs2165739 and/or rs10508021 for use in predicting that an individual has a risk of susceptibility for SLE and/or for MS.

In another aspect the invention relates to a combination of rs10516486 with rs950357, rs1342337, or rs1937840; or rs10516483 with rs1401385, rs1717045, rs1478895, rs1049380, rs10507393, or rs10508021; or rs1872701 with rs10508021; or rs10516483 with rs1478895 and rs1049830 for use in predicting that an individual has a risk of susceptibility for SLE.

The invention further relates to a method for predicting a risk of susceptibility for SLE and/or for MS in an individual comprising:

a. using the nucleic acid extracted from a sample of said individual;
b. identifying the presence of a useful genetic marker in said individual by known methods;
c. based on the results of step b) making a prediction of the probability as to the susceptibility for SLE and/or MS for said individual.

In preferred embodiments of the method according to the invention the genetic marker is one or more SNPs selected from the group consisting of rs10516486, rs10516483, rs1872701, rs10496637, rs950357, rs10516928, rs1342337, rs1937840, rs10505774, rs2302733, rs738981, rs6683832, rs2300166, rs1901765, rs1401385, rs1717045, rs790837, rs10484396, rs10485136, rs9294364, rs881278, rs720613, rs1478895, rs1992529, rs2289965, rs10502263, rs1049380, rs10506140, rs10507393, rs10508021, rs1886560 and rs2165739, SNPs in Linkage Disequilibrium (LD) with one or more of these SNPs, and one or more SNPs in LD with either of BANK1, BLK and/or ITPR2 genes.

In said method it could be shown that particularly useful in a preferred embodiment is a method wherein the genetic marker is a combination of the SNPs selected from rs10516486 combined with rs10496637, rs950357, rs10516928, rs1342337, rs1937840, rs10505774, rs2302733 and/or rs738981; or rs10516483 combined with rs6683832, rs2300166, rs1901765, rs1401385, rs1717045, rs790837, rs10484396, rs10485136, rs9294364, rs881278, rs720613, rs1478895, rs1992529, rs2289965, rs10502263, rs1049380, rs10506140, rs10507393, rs10508021 and/or rs1886560; or rs1872701 combined with rs2165739 and/or rs10508021; or a combination of the above combinations.

Even more preferred is a method wherein the genetic marker is a combination of rs10516483, rs1478895 and rs1049380, or SNPs in LD with these SNPs, or with either of BANK1, BLK and/or ITPR2 genes.

The invention further relates to a method for predicting a risk of susceptibility for SLE in an individual comprising:

a. using the nucleic acid extracted from a sample of said individual;
b. identifying the presence of a useful genetic marker in said individual by known methods, wherein the genetic marker is a combination of rs10516486 with rs950357, rs1342337, or rs1937840; or rs10516483 with rs1401385, rs1717045, rs1478895, rs1049380, rs10507393, or rs10508021; or rs1872701 with rs10508021; or rs10516483 with rs1478895 and rs1049830; or SNPs in LD with either of BANK1, BLK and/or ITPR2 genes; and
c. based on the results of step b. making a prediction of the probability as to the susceptibility for SLE for said individual.

Preferably, in said method the genetic marker is a combination of rs10516483, rs1478895 and rs1049380, or SNPs in LD with either of BANK1, BLK and/or ITPR2 genes.

### EXAMPLES

In order to achieve the invention, data from a systemic lupus erythematosus (SLE) genome-wide association scan (GWAS)¹ were used and searched for epistatic interactions (epistatic scan). For this purpose we developed a genotypic interaction method based on contingency tables for all possible genotype combinations between pairs of SNPs with r²<0.80. We then calculated a Pearson S score of interaction association and its chi-squared p value. To compute epistasis each observed interacting combination was tested against the hypothesis of independence to derive an epistasis score (Sₑ) and a p value was obtained through permutation (Epistatic scan methodology).

Out of 112,463 SNPs, 13,008 tag SNPs were selected for analysis (4,897 in LD blocks and 8,111 isolates) with 84,597,528 interactions tested. Applying cutoff thresholds of 1e⁻⁵ for the association p-value and 1e⁻³ for epistatic p-values as described (Epistatic scan methodology) we selected 1,626 SNP interactions involving 1,206 distinct SNPs. Those SNPs were mapped to genes on the NCBI Build 36 genome sequence and a sub-network of 497 gene interactions involving 418 genes was created. The obtained genetic interaction network displayed a scale-free topological property, with 60% of the genes involved in one interaction 17% in two and 6 genes (“hubs”) involved in >20 interactions. Among the most connected hub genes BANK1 was involved in 30 associated and epistatic genetic interactions (Table 1). We recently identified BANK1 as a gene associated with SLE, a complex, autoimmune disease¹. BANK1 is exclusively expressed in B cells, making this a gene of relevance in disease pathogenesis.

We focused on two genes with which BANK1 showed interaction, BLK, also found to be associated with SLE in two GWAS^{2,3} and expressed in B cells and ITPR2, one of the ITPR genes that codes for the IP3R calcium channel an ubiquitous protein inducing calcium mobilization from the endoplasmic reticulum stores to the cytosol upon binding to BANK1⁴. The interaction between BLK and BANK1 had an epistatic OR (Odds Ratio)=2.38 (95% c.i. 1.69-3.36; 35% in cases vs 18% in controls). The strongest interaction between BANK1 and ITPR2 had an epistatic OR=2.49 (c.i. 1.66-3.73; 23% in cases vs 11% in controls). We also observed an associated and epistatic genetic interaction between BANK1, ITPR2 and BLK with epistatic odds ratios of OR=3.20 (95% c.i. 2.04-5.01; 21% in cases vs 8% in controls; S=27.6; P=1.5×10⁻⁷; Sₑ=14.67, P_{eb}<0.0002) (FIG. 1).

We replicated the interactions using two independent sets of cases and controls comprising over 4,000 individuals (Table 2). A meta-analysis showed an interaction between BANK1 and ITPR2 of P=3.6×10⁻⁶ and between BANK1 and BLK of P=4.11×10⁻¹¹. However the epistatic score (Se) did not reach significance suggesting that more interacting genes are to be identified. More importantly, not all SNPs within each gene were involved in the interaction. For instance, despite having over 58 SNPs genotyped across BLK, the only interacting and epistatic SNPs were located in the 5′UTR and promoter region of the gene represented by SNPs rs13277113 and rs12680762, both associated with SLE^{2,3}. In BANK1 rs10516487 leading to a R61H change in exon 2′ was the primary SNP involved in the epistasis together with SNP rs10516483. In ITPR2, SNPs found in the 3′UTR showed interaction with BANK1. We therefore tested if the interacting SNPs of ITPR2 correlated with differential levels of ITPR2 mRNA. Indeed, two of the SNPs in the 3′ UTR of ITPR2 (rs1049380 and rs4654) correlate with expression levels of this gene while a SNP outside the 3′ UTR region of ITPR2 did not correlate with transcript levels of ITPR2 (FIG. 2 and FIG. 3). The protein interaction between the products of BANK1 and ITPR2 is known⁴ and the BANK1 protein contains an IP3R-binding domain. Conversely, physical interaction of BANK1 and BLK is not known. BANK1 co-precipitated with BLK (FIG. 4), potentially through the Src-tyrosine kinase-binding domain to which LYN also binds^{5,6}. Also, in cells co-transfected with BANK1 and BLK-GFP a clear co-localization of BLK and BANK1 within cytoplasmic vesicles was observed, while BLK but not BANK1 localized also in the cell membrane (FIG. 5). Our results overall reveal a novel protein interaction between BANK1 and BLK and further show that BANK1, in its adaptor role is partly retaining BLK within cytoplasmic vesicles.

We developed a method to detect genetic interaction and epistasis based on genotypes and testing basically dominant and recessive models. The interactions identified here were not clearly reproduced using logistic regression analysis with PLINK⁷, as such analysis only relies on alleles and is probably less powerful in detecting non-additive epistatic interactions.

We further show that the genetically-interacting genes also encode physically-interacting proteins revealing a novel disease pathway of importance in the pathogenesis of SLE where the independent effects of each of the genes synergize in an epistatic effect with significantly more important contributions in disease susceptibility than the effects of the individual genes. Some of the genes potentially interacting with BANK1 are also involved in the type I interferon pathway of genes, shown to be of major importance in disease pathogenesis⁸⁻¹¹. Indeed, we observe that in PBMCs BANK1 is induced with IFNa while BLK is down-regulated, suggesting a potential bridge between the innate immune system and BcR-mediated activation (FIG. 6).

Most of the major genes identified for most complex diseases, including lupus, did not show genetic interaction among them and interactions identified to date have not been confirmed^{3,12,13}, least at the protein level. The finding of the invention indicate that each of these major genes for lupus represents each a pathogenic pathway of importance in some individuals. In the present study we observe that approximately one fourth of all individuals with lupus (21%) had risk genotypes for the interacting genes. It is possible that most lupus genetic susceptibility can be explained by a variable number of interacting genes within 4-5 distinct pathways represented by a few major genes (i.e. HLA, IRF5, ITGAM, STAT4 for lupus) with additive effects and that such pathways define the pathogenic process in those individuals. The findings of the present invention represent the first epistatic genetic interactions described and replicated in a complex disease, involving interacting proteins and defining pathways of disease pathogenesis.

### Materials

Patients and controls used for the 100 k GWAS have been described previously. Two completely independent sets of cases and controls were used. The first set comprises SLE cases and sex, age and ethnicity matched controls from a multicenter collection in Europe all of which have been previously described. The second set. All cases fulfilled the 1982 classification criteria for SLE.

### Genotyping

The genotyping of the 100 k array has been described. Genotyping of the first replication sets for BANK1, BLK and ITPR2 was performed for SNPs rs10516487, rs10516483, rs1478895, rs1049380, rs4654, rs1994484. SNPs using the assay-on-demand TaqMan ABI system, with the exception of set 2 where BANK1 and BLK were genotyped on the BeadExpress Illumina system for SNPs covering the complete genes. This genotyping was performed at the Oklahoma Medical Research Foundation while the TaqMan genotyping was performed at the Rudbeck Laboratory at Uppsala University and at the Instituto de Biomedicina y Parasitología López-Neyra in Granada, pain (for Spanish samples). Only samples having less than 5% genotyping calls were used for the analyses.

### Epistatic Scan Methodology

SNP selection

SNPs from the 100 k genome-wide association scan were first quality controlled: Hardy-Weinberg Equilibrium (HWE) in controls p<0.01 and maximum missing data rate per SNP<5%. Only frequent markers were kept for analysis: minimum allele frequencies 30% in controls and 10% in cases, and minimum genotype frequencies 10% in controls and 5% in cases. Then genome-wide Linkage Disequilibrium (LD) blocks were determined using the method of Gabriel et al. (18) and tag SNPs were selected (one random SNP per LD block and all SNPs not in LD blocks) thereby.

### Genetic Interaction Association

For every couple of SNPs that are not in LD (r²<0.8), the co-occurrences of genotype counts are recorded in a 2×9 contingency table (2 rows: cases/controls; 9 columns corresponding to the 9 possible genotype combinations, i.e. a 3×3 table): T=[cₖᵢⱼ] where cₖᵢⱼ represents the number of patients in cases (k=0) or controls (k=1) having i copies of the first SNP minor allele (i=0, 1, 2) and jcopies of the second SNP minor allele (j=0, 1, 2). From this table, we derive eight 2×2 contingency tables, representing combinations of dominant and recessive models: Let a/A and b/B be the alleles of both SNPs, each 2×2 contingency table contains respectively the counts in cases of aa/bb (c₀₀₀), aa/BB (c₀₀₂), AA/bb (c₀₂₀), AA/BB (c_{O22}), aa+aA/bb+bB (c₀₀₀+c₀₀₁+c₀₁₀+c₀₁₁), aa+aA/bB+BB (c₀₀₁c₀₀₂+c₀₁₁+c₀₁₂), aA+AA/bb+bB (c₀₁₀+c₀₁₁+c₀₂₀+c₀₂₁), aA+AA/bB+BB (c₀₁₁+c₀₁₂+c₀₂₁+c₀₂₂) in the upper left cell, the similar count in controls in the lower left cell and the complement counts in cases and controls in the upper and lower right cells respectively. For each such 2×2 contingency table, a Pearson score Sₜ (t=1.8) is computed and the p-value pₜ is approximation using a c² distribution assumption with one degree of freedom (df).

### Estimation of the Epistatic Effect

For every couple of SNP, a 2×9 contingency table under the hypothesis of independency between both SNPs (no epistasis) is derived: T⁰=[c⁰ₖᵢⱼ], c⁰ₖᵢⱼ=(cₖ₀ⱼ+cₖ₁ⱼ+cₖ₂ⱼ)(cₖᵢ₀+cₖᵢ₁+cₖᵢ₂)/nₖ where nₖ is the total number of patients in cases (k=0) or controls (k=1). Similarly as above, eight 2×2 contingency tables are derived and eight Pearson scores are computed: S″ₜ (t=1.8). The epistatic score is defined as follows:

S^{e}ₜ=Sₜ−S⁰ₜ

This score is the difference of two dependent scores, each one following asymptotically a 1−df c². Therefore it does not follow any known statistical law and p-values p^{e}ₜ have to be empirically determined by permutations.

### Gene Expression Analysis

### RNA Purification and Expression Analysis of the Genes

Total RNA was purified with TRIZOL Reagent (Invitrogen) from peripheral blood mononuclear cells (PBMCs) obtained with agreed consent from healthy donors. 2 μg of RNA was reverse-transcribed with 2 U of MuLV transcriptase in buffer containing 5 mM MgCl2, 1 mM dNTPs, 0.4 U of RNase inhibitor and 5 μM oligo-dT. All reagents were purchased from Applied Biosystems. cDNA synthesis was performed at 42° C. for 80 min, and then the reaction was terminated at 95° C. for 5 min. BANK1, BLK, and ITPR2 expression was determined by quantitative real-time PCR on 7900 HT Sequence Detector (Applied Biosystems) with SDS 2.2.2 software using SYBR Green for signal detection. The following primer pairs were used: for

| Primer | Sequence | SEQ ID NO. |
|---|---|---|
| full-length BANK1 | 5′-TCAAAGCAGATGGGAGATCTCAAC-3′ | 33 |
| isoform forward primer | | |
| full-length BANK1 | 5′-CACATGGAATTTCAGTGGGAAGCAC-3′ | 34 |
| isoform reverse primer | | |
| BLK forward primer | 5′-ACGGCCCAAGAGGGGGCCAAGT-3′ | 35 |
| BLK reverse primer | 5′-GTTGCTCATCCCTGGGTATGGCA-3′; | 36 |
| ITPR2 forward primer | 5′-TGGCTCAAATGATTGTGGAGAAGAAT-3′ | 37 |
| ITPR2 reverse primer | 5′-ACTGATGAAAGGCTAGTCACGGCTTC-3′ | 38 |

We performed initial denaturation at 95° C. for 5 min followed by 45 cycles of PCR (95° C. for 15 s, 62° C. for 10 s and 72° C. for 15 s). PCR buffer provided with enzyme was supplemented with 3 mM MgCl2, 200 μM of each of dNTPs, primers, SYBR Green (Molecular Probes), 15 ng of cDNA and 0.5 U of Platinum Taq polymerase (Invitrogen). Expression levels were normalized to the levels of TBP in the same samples using comparative 2-ΔCt-method and amplified with commercial reagents (Applied Biosystems). All experiments were run in triplicate. Independent cDNA synthesis was carried out twice. Statistical calculations were performed with available on-line GraphPad Software using two-tailed t-test.

### Cloning and Expression Constructs

BANK1 and BLK sequences were amplified by PCR using cDNAs from human blood and BJAB cell line respectively. The open reading frames were cloned in pcDNA3.1D/V5-His (Invitrogen) and confirmed by sequencing. Proteins tagged by V5 and His epitopes at the C-terminal were produced by deletion of the stop codons. The N-terminal FLAG-tagged BANK plasmids were constructed by sequential PCR using overlapping primers. The amplified product coding for flag fused to BANK1 variants was cloned into pCR4-TOPO (Invitrogen) excised by EcoRI and BamHI and directional sub-cloned into pIRESS2-EGFP (Clontech):

| Construct | Name | Sequence | SEQ ID NO. |
|---|---|---|---|
| pcDNA-BLK-v5 | f-BLK | 5′-CACCatggggctggtaagtagc-3′ | 39 |
| | r-BLK | 5′-gggctgcagctcgtactgcc-3′ | 40 |
| pcDNA-BANK | f-BANK | 5′-CACCatgctgccagcagcgccag-3′ | 41 |
| | r-BANK | 5′-ataataaccttctttaatgatctttcttgc-3′ | 42 |
| plRES-Flag-BANK | f-FLAG-k | 5′-cacaaccatggattacaaggatgacgacg-3′ | 43 |
| | f-FLAG-m | 5′-attacaaggatgacgacgataagatgctgc-3′ | 44 |
| | f-FLAG-BANK | 5′-cgacgataagatgctgccagcagcgccag-3′ | 45 |
| | r-BANK-H1 | 5′-AGGATccttctttaatgatctttc-3′ | 46 |

Note:
Bases modified for cloning are indicated in uppercase and the start codons in bold.

### Antibodies

A synthesized peptide with the sequence ETKHSPLEVGSESSC was used to immunize rabbits to generate polyclonal BANK1 anti-sera (ET-BANK). The sera was affinity purified against the peptide using the SulfoLink Kit (Pierce). Additional antibodies used in this study include an anti-mouse and anti-rabbit Alexa Fluor 488, anti-mouse and anti-rabbit Alexa Fluor 647, anti-V5 (Invitogen); anti-Flag M2 monoclonal and rabbit anti-Flag (Sigma); anti-rabbit and anti-mouse IgG HRP (Zymed).

### Co-Immunoprecipitation and Immunoblot

Cells were seeded on 6-well plates and transfected with a total of 4 ug expression plasmids using Lipofectamine 2000. 40 h after transfection cells were solubilized in Triton X-100 buffer (1% Triton X-100, 50 mM HEPES pH 7.1, 150 mM Nacl, 1 mM EDTA, 2 mM Na3VO4, 10 Glycerol, 0.1% SDS) containing protease inhibitors (Roche) and 1 mM PMSF. Aliquots of the pre-cleared lysates were saved for input analysis and the rest of the lysate was incubated sequentially with rabbit anti-Flag and immobilized A-Sepharose beads (GE Heathcare). The beads were washed five times with PBS and the immunoprecipitates were eluted with SDS sample buffer by boiling 5 min. SDS-PAGE and immmunoblotting were carried out using standard protocols. (Loaded wells for the IP correspond to ⅖ of the initial cell extract while wells for the cell lysate contain 1/40 of the original cell extract).

### Confocal Microscopy

Transfected cells were fixed at room temperature for 20 minutes with 3,7% paraformaldehyde in PBS/0.18% Triton-X and permeabilized in ice-cold 50:50 methanol-acetone at −20° C. for 10 minutes. After blocking in 3% BSA, 3% goat serum in PBT the antibodies were diluted in blocking buffer and incubated overnight at 4° C. Fluorochrome-conjugated secondary antibodies were incubated for 2 hours at room temperature and counterstained with SlowFade antifade with DAPI (Invitrogen). Confocal microscopy was performed using a Zeiss 510 Meta confocal scanning microscope. Dual- or triple-color images were acquired by consecutive scanning with only 1 laser line active per scan to avoid cross-excitation.

TABLE 1
Associated and epistatic interactions involving BANK1 SNPs.

| BANK1 | Position | Alleles | | Assoc. | Interacting | | | | Alleles | | Assoc. | Risk SNP |
|---|---|---|---|---|---|---|---|---|---|---|---|---|
| SNP | on chr. 4 | (a) | | p-value | SNP | Gene | Chr. | Position | (a) | | p-value | combination |
| rs10516486 | 103,108,454 | C | T | 1.5E−03 | rs10496637 | CNTNAP5 | 2 | 125,023,420 | T | G | 2.2E−02 | T & G (CC \| TT) |
| | | | | | rs950357 | SIDT1 | 3 | 114,816,923 | A | C | 7.7E−02 | CC & AA |
| | | | | | rs10516928 | GRID2 | 4 | 94,909,484 | T | G | 6.3E−03 | T & G (CC \| TT) |
| | | | | | rs1342337 | KCNQ5 | 6 | 73,692,956 | A | G | 6.5E−03 | CC & AA |
| | | | | | rs1937840 | AKR1C3 | 10 | 5,131,307 | C | G | 2.4E−02 | CC & CC |
| | | | | | rs10505774 | EMP1 | 12 | 13,327,672 | C | T | 3.5E−01 | T & T (CC \| CC) |
| | | | | | rs2302733 | PRDM4 | 12 | 106,656,666 | A | G | 1.8E−01 | T & G (CC \| AA) |
| | | | | | rs738981 | FBXO7 | 22 | 31,211,339 | C | T | 1.1E−01 | T & T (CC \| CC) |
| rs10516483 | 103,149,083 | C | G | 5.7E−04 | rs6683832 | ATG4C | 1 | 62,988,925 | G | A | 1.0E+00 | G & A (CC \| GG) |
| | | | | | rs2300166 | PTGER3 | 1 | 71,166,988 | G | A | 1.1E−01 | G & A (CC \| GG) |
| | | | | | rs1901765 | RNF144 | 2 | 7,168,421 | T | C | 1.1E−01 | G & C (CC \| TT) |
| | | | | | rs1401385 | ST3GAL5 | 2 | 86,036,824 | T | A | 2.1E−02 | CC & TT |
| | | | | | rs1717045 | DPP10 | 2 | 115,888,863 | A | G | 9.1E−01 | CC & AA |
| | | | | | rs790837 | CTXN3 | 5 | 127,032,405 | G | A | 4.3E−02 | G & A (CC \| GG) |
| | | | | | rs10484396 | ZNF184 | 6 | 27,505,177 | T | C | 1.3E−03 | G & C (CC \| TT) |
| | | | | | rs10485136 | FAM83B | 6 | 54,864,454 | T | C | 1.6E−01 | G & C (CC \| TT) |
| | | | | | rs9294364 | CGA | 6 | 87,847,548 | G | A | 9.7E−02 | G & A (CC \| GG) |
| | | | | | rs881278 | MYCT1 | 6 | 153,084,200 | A | G | 2.0E−01 | G & G (CC \| AA) |
| | | | | | rs720613 | POT1 | 7 | 124,046,621 | C | T | 6.6E−03 | G & T (CC \| CC) |
| | | | | | rs1478895 | BLK | 8 | 11,390,744 | C | G | 4.4E−03 | CC & CC |
| | | | | | rs1992529 | MBL2 | 10 | 54,174,055 | C | T | 9.4E−02 | G & T (CC \| CC) |
| | | | | | rs2289965 | IGSF22 | 11 | 18,685,226 | C | T | 9.7E−01 | G & T |
| | | | | | rs10502263 | BRCC2 | 11 | 121,447,531 | T | C | 5.8E−01 | G & C (CC \| TT) |
| | | | | | rs1049380 | ITPR2 | 12 | 26,380,811 | T | G | 1.1E−02 | CC & TT |
| | | | | | rs10506140 | SLC2A13 | 12 | 38,636,641 | G | A | 3.2E−01 | G & A (CC \| GG) |
| | | | | | rs10507393 | ALOX5AP | 13 | 30,225,521 | T | C | 1.1E−01 | CC & TT |
| | | | | | rs10508021 | ABCC4 | 13 | 94,692,121 | G | C | 1.1E−02 | CC & GG |
| | | | | | rs1886560 | HS6ST3 | 13 | 95,876,553 | C | T | 2.8E−01 | G & T (CC \| CC) |
| rs1872701 | 103,310,859 | G | T | 2.5E−01 | rs2165739 | NCOA1 | 2 | 24,728,455 | A | G | 5.0E−03 | G & T |
| | | | | | rs10508021 | ABCC4 | 13 | 94,692,121 | G | C | 1.1E−02 | GG & GG |

| BANK1 | Position | Alleles | | Assoc. | Interacting | Frequency in | Frequency in | Odds ratio |
|---|---|---|---|---|---|---|---|---|
| SNP | on chr. 4 | (a) | | p-value | SNP | cases | controls | [95% c.i.] |
| rs10516486 | 103,108,454 | C | T | 1.5E−03 | rs10496637 | 20% | 35% | 0.45 [0.32-0.64] |
| | | | | | rs950357 | 27% | 13% | 2.44 [1.69-3.54] |
| | | | | | rs10516928 | 28% | 46% | 0.47 [0.34-0.65] |
| | | | | | rs1342337 | 28% | 15% | 2.32 [1.61-3.35] |
| | | | | | rs1937840 | 27% | 13% | 2.36 [1.62-3.42] |
| | | | | | rs10505774 | 25% | 41% | 0.48 [0.35-0.67] |
| | | | | | rs2302733 | 27% | 44% | 0.48 [0.35-0.67] |
| | | | | | rs738981 | 20% | 37% | 0.43 [0.30-0.61] |
| rs10516483 | 103,149,083 | C | G | 5.7E−04 | rs6683832 | 44% | 61% | 0.50 [0.37-0.68] |
| | | | | | rs2300166 | 32% | 50% | 0.46 [0.34-0.64] |
| | | | | | rs1901765 | 26% | 44% | 0.46 [0.33-0.63] |
| | | | | | rs1401385 | 24% | 11% | 2.63 [1.76-3.92] |
| | | | | | rs1717045 | 26% | 13% | 2.43 [1.66-3.56] |
| | | | | | rs790837 | 30% | 48% | 0.47 [0.34-0.64] |
| | | | | | rs10484396 | 43% | 63% | 0.45 [0.33-0.61] |
| | | | | | rs10485136 | 48% | 66% | 0.47 [0.35-0.64] |
| | | | | | rs9294364 | 26% | 42% | 0.47 [0.34-0.66] |
| | | | | | rs881278 | 42% | 59% | 0.50 [0.37-0.68] |
| | | | | | rs720613 | 20% | 38% | 0.42 [0.30-0.60] |
| | | | | | rs1478895 | 35% | 18% | 2.38 [1.69-3.36] |
| | | | | | rs1992529 | 42% | 60% | 0.47 [0.35-0.64] |
| | | | | | rs2289965 | 45% | 62% | 0.49 [0.36-0.66] |
| | | | | | rs10502263 | 30% | 47% | 0.48 [0.35-0.66] |
| | | | | | rs1049380 | 23% | 11% | 2.49 [1.66-3.73] |
| | | | | | rs10506140 | 43% | 61% | 0.47 [0.35-0.65] |
| | | | | | rs10507393 | 23% | 11% | 2.57 [1.72-3.85] |
| | | | | | rs10508021 | 23% | 11% | 2.48 [1.65-3.72] |
| | | | | | rs1886560 | 31% | 48% | 0.49 [0.36-0.67] |
| rs1872701 | 103,310,859 | G | T | 2.5E−01 | rs2165739 | 37% | 53% | 0.51 [0.38-0.69] |
| | | | | | rs10508021 | 22% | 10% | 2.45 [1.64-3.67] |

(a) The risk allele is reported in the first column, the other allele in the second allele. ‘?’ means that both alleles have similar frequencies in cases and controls.

TABLE 2
Summary of the SNP/gene Interactions Between BANK1- BLK, BANK1-ITPR2
and BLK-ITPR2 in Three Independent Sets of Cases and Controls.

| BANK1 | Genotype | ITPR2 | Genotype | | OR | OR_low | OR_high | P^{§} | Se* | f_cases** | f_ctrls | N |
|---|---|---|---|---|---|---|---|---|---|---|---|---|
| rs10516483 | CC | rs1049380 | AA | Set 1 (100k) | 2.49 | 1.66 | 3.73 | 6.35E−06 | 8.5 | 23% | 11% | 758 |
| | | | | Set 2 (USA) | na | na | na | na* | na | na | na | 0 |
| | | | | Set 3 (Europe) | 1.27 | 1.05 | 1.53 | 6.55E−03 | na | 18% | 15% | 3103 |
| | | | | Meta-analysis | na | na | na | na | na | na | na | 3861 |
| rs10516487 | GG | rs1049380 | AA | Set 1 (100k) | 1.73 | 1.23 | 2.42 | 1.51E−03 | 0.5 | 30% | 20% | 781 |
| | | | | Set 2 (USA) | 1.07 | 0.84 | 1.35 | 0.577 | −0.7 | 26% | 25% | 1469 |
| | | | | Set 3 (Europe) | 1.16 | 0.99 | 1.38 | 5.72E−02 | na | 30% | 28% | 2675 |
| | | | | Meta-analysis | 1.20 | 1.06 | 1.36 | 1.99E−03 | na | | | 4925 |
| rs10516487 | G | rs1049380 | A | Set 1 (100k) | 1.66 | 1.09 | 2.53 | 1.79E−02 | 1.6 | 87% | 81% | 781 |
| | | | | Set 2 (USA) | 1.58 | 1.17 | 2.14 | 2.50E−03 | −0.3 | 88% | 83% | 1469 |
| | | | | Set 3 (Europe) | 1.37 | 1.10 | 1.72 | 3.94E−03 | na | 88% | 83% | 2675 |
| | | | | Meta-analysis | 1.48 | 1.25 | 1.74 | 1.19E−06 | na | | | 4925 |
| BANK1 | | BLK | | | | | | | | | | |
| rs10516483 | CC | rs1478895 | CC | Set 1 (100k) | 2.38 | 1.69 | 3.36 | 4.83E−07 | 8.9 | 35% | 18% | 763 |
| | | | | Set 2 (USA) | na | na | na | na | na | na | na | 0 |
| | | | | Set 3 (Europe) | 1.41 | 1.25 | 1.59 | 1.72E−05 | na | 26% | 19% | 250 |
| | | | | Meta-analysis | na | na | na | na | na | na | na | 3283 |
| rs10516487 | GG | rs1478895 | CC | Set 1 (100k) | 1.82 | 1.35 | 2.45 | 8.27E−05 | 3.7 | 48% | 33% | 788 |
| | | | | Set 2 (USA) | 1.29 | 1.04 | 1.60 | 2.09E−02 | 2.2 | 37% | 31% | 1486 |
| | | | | Set 3 (Europe) | 1.37 | 1.18 | 1.59 | 6.58E−05 | na | 44% | 36% | 2248 |
| | | | | Meta-analysis | 1.41 | 1.25 | 1.57 | 5.53E−10 | na | | | 4522 |
| rs10516487 | G | rs1478895 | C | Set 1 (100k) | 1.72 | 1.00 | 1.70 | 4.76E−02 | −0.1 | 93% | 89% | 788 |
| | | | | Set 2 (USA) | 1.68 | 1.20 | 1.68 | 2.52E−03 | 0.6 | 92% | 87% | 1486 |
| | | | | Set 3 (Europe) | 1.46 | 1.09 | 1.95 | 9.02E−03 | na | 93% | 87% | 2248 |
| | | | | Meta-analysis | 1.57 | 1.28 | 1.93 | 6.66E−06 | na | | | 4522 |
| ITPR2 | | BLK | | | | | | | | | | |
| rs1049380 | TT | rs1478895 | CC | Set 1 (100k) | 1.61 | 1.19 | 1.53 | 2.16E−03 | −1.6 | 41% | 30% | 781 |
| | | | | Set 2 (USA) | 1.07 | 0.87 | 1.32 | 0.525 | 0.4 | 38% | 37% | 1473 |
| | | | | Set 3 (Europe) | 0.86 | 0.74 | 1.01 | 5.67E−02 | na | 37% | 38% | 2666 |
| | | | | Meta-analysis | 1.01 | 0.90 | 1.14 | 0.852 | na | | | 4920 |
| BANK1 | rs1051648 | GG | | Set1, set2, set3 | 1.41 | 1.26 | 1.57 | 3.35E−10 | | 57% | 49% | 5476 |
| | rs1051648 | G | | Set1, set2, set3 | 1.69 | 1.36 | 2.09 | 7.13E−02 | | 94% | 91% | 5476 |
| ITPR2 | rs1049380 | AA | | Set1, set2, set3 | 0.94 | 0.84 | 1.04 | 0.245 | | 52% | 53% | 5775 |
| | rs1049380 | A | | Set1, set2, set3 | 1.14 | 0.94 | 1.37 | 0.620 | | 93% | 92% | 5775 |
| BLK | rs1478895 | CC | | Set1, set2, set3 | 1.15 | 1.02 | 1.29 | 9.23E−02 | | 74% | 71% | 5843 |
| | rs1478895 | C | | Set1, set2, set3 | 1.14 | 0.82 | 1.59 | 0.866 | | 98% | 97% | 5843 |

na: not analyzed; rs10516483 was not genotyped in the USA set
Set 2: European-American set
Set 3: The combined set of German, Italian, Argentine and Spanish cases and controls
^{§}For individual sets a Pearson P value was computed; For the meta-analysis a Mantel-Haenszel p value is provided
*Se is the epistasis score (See Epistatic scan methodology)
**The frequency refers to the presence of the allele as a count of individuals

### REFERENCES

1. Kozyrev, S. V., Abelson, A. K., Wojcik, J., Zaghlool, A., Ling a Reddy, M. V., Sanchez, E. et al. Nat Genet 40, 211-6 (2008).
2. Horn, G., Graham, R. R., Modrek, B., Taylor, K. E., Ortmann, W., Garnier, S. et al. N Engl J Med 358, 900-9 (2008).
3. Harley, J. B., Alarcon-Riquelme, M. E., Criswell, L. A., Jacob, C. O., Kimberly, R. P., Moser, K. L. et al. Nat Genet 40, 204-10 (2008).
4. Yokoyama, K., Su Ih, I. H., Tezuka, T., Yasuda, T., Mikoshiba, K., Tarakhovsky, A. et al. Embo J 21, 83-92 (2002).
5. Xu, Y., Harder, K. W., Huntington, N. D., Hibbs, M. L. & Tarlinton, D. M. Immunity 22, 9-18 (2005)
6. Sefton, B. M. & Taddie, J. A. Curr Opin Immunol 6, 372-9 (1994).
7. Purcell, S., Neale, B., Todd-Brown, K., Thomas, L., Ferreira, M. A., Bender, D. et al. Am J Hum Genet 81, 559-75 (2007).
8. Banchereau, J. & Pascual, V. Immunity 25, 383-92 (2006).
9. Bennett, L., Palucka, A. K., Arce, E., Cantrell, V., Borvak, J., Banchereau, J. et al. J Exp Med 197, 711-23 (2003).
10. Blanco, P., Palucka, A. K., Gill, M., Pascual, V. & Banchereau, J. Science 294, 1540-3 (2001).
11. Alarcon-Riquelme, M. E. Nat Genet 38, 866-7 (2006).
12. Prescott, N. J., Fisher, S. A., Franke, A., Hampe, J., Onnie, C. M., Soars, D. et al. Gastroenterology 132, 1665-71(2007).
13. van Heel, D. A., Dechairo, B. M., Dawson, G., McGovern, D. P., Negoro, K., Carey, A. H. et al. Hum Mol Genet 12, 2569-75 (2003).
14. Jordan, M. S., Singer, A. L. & Koretzky, G. A. (2003) Nat Immunol 4, 110-6.
15. Kurosaki, T. (2002) Nat Rev Immunol 2, 354-63.
16. Okada, T., Maeda, A., Iwamatsu, A., Gotoh, K. & Kurosaki, T. (2000) Immunity 13, 817-27.
17. Patterson, R. L., Boehning, D. & Snyder, S. H. (2004) Annu Rev Biochem 73, 437-65.
18. Gabriel S B, Schaffner S F, Nguyen H, Moore J M, Roy J, Blumenstiel B, Higgins J, DeFelice M, Lochner A, Faggart M, Liu-Cordero S N, Rotimi C, Adeyemo A, Cooper R, Ward R, Lander E S, Daly M J, Altshuler D (2002) The structure of haplotype blocks in the human genome. Science 296:2225-2229

1-10. (canceled)

11. A method for genotyping comprising the steps of:
a) using a nucleic acid isolated from a sample of an individual;
b) determining the type of nucleotide in:
rs10516486 and rs950357,
rs10516486 and rs1342337,
rs10516486 and rs1937840,
rs10516483 and rs1401385,
rs10516483 and rs1717045,
rs10516483 and rs1478895,
rs10516483 and rs1049380,
rs10516483 and rs10507393,
rs10516483 and rs10508021,
rs1872701 and rs10508021, or
rs10516483, rs1478895 and rs1049830 in the diallelic marker, or in a SNP in Linkage Disequilibrium (LD) with one or more of these SNPs or one or more SNP in LD with either of BANK1 BLK and/or ITPR2; and
c) correlating the results of step b) with a risk of susceptibility for Systemic Lupus Erythematosus (SLE).

12. The method according to claim 11, wherein the identity of the nucleotides at said diallelic markers is determined for both copies of said diallelic markers present in said individual's genome.

13. The method according to claim 11, wherein said determining is performed by a microsequencing assay.

14. The method according to claim 11, further comprising amplifying a portion of a sequence comprising the diallelic marker prior to said determining step.

15. The method according to claim 14, wherein said amplifying is performed by PCR.

16. The method according to claim 11, wherein the presence of a C or a T in rs1.0516486, a C or a G in rs10516483, a G or a T in rs1872701, an A or C in rs950357, an A or a G in rs1342337, a C or a G in rs1937840, a T or an A in rs1401385, an A or a G in rs1717045, a C or a G in rs1478895, a T or a G in rs1049380, a T or a C in is 10507393, and/or a G or a C in rs10508021, in said individual indicates that said individual has a risk of susceptibility to SLE, wherein the risk allele is listed first.

17. A composition comprising at least two SNPs selected from the group consisting of rs10516486, rs950357, rs1342337, rs1937840, rs10516483, rs1401385, rs1717045, rs1478895, rs1049380, rs10507393, rs10508021, rs1872701, SNPs in Linkage Disequilibrium (LD) with one or more of these SNPs, and one or more SNPs in LD with either of BANK1, BLK and/or ITPR2 for use in predicting that an individual has a risk of susceptibility for SLE.

18. A method for predicting a risk of susceptibility for SLE in an individual comprising:
a) using the nucleic acid extracted from a sample of said individual;
b) identifying the presence of a useful genetic marker in said individual by known methods, wherein the genetic marker is a combination of rs10516486 with rs950357, rs1342337, or rs1937840; or rs10516483 with rs1401385, rs1717045, rs1478895, rs1049380, rs10507393, or rs10508021; or rs1872701 with rs10508021; or rs10516483 with rs1478895 and rs1049830; or SNPs in LD with either of BANK1, BLK and/or ITPR2 genes; and
c) based on the results of step b) making a prediction of the probability as to the susceptibility for SLE for said individual.

19. The method according to claim 18, wherein the genetic marker is a combination of rs10516483, rs 1478895 and rs 1049380, or SNPs in LD with either of BANK1, BLK and/or ITPR2 genes.
